(19) United States
(12) Patent Application Publication
(10) Pub. No.: US 2014/0016667 A1
(43) Pub. Date: Jan. 16, 2014

(54) METHOD FOR MONITORING WATER TEMPERATURE

(71) Applicant: Blue Water Satellite, Inc., Bowling Green, OH (US)

(72) Inventor: Louis M. Sanderson, Perrysburg, OH (US)

(21) Appl. No.: 13/941,591

(22) Filed: Jul. 15, 2013

Related U.S. Application Data
(60) Provisional application No. 61/671,410, filed on Jul. 13, 2012.

(51) Int. Cl. G01N 33/18

(52) U.S. Cl. CPC G01N 33/18
USPC 374/121; 345/440

(57) ABSTRACT

Methods are provided for monitoring water temperature using light reflected therefrom, where measurement of reflected light can be obtained from satellite imagery. Such methods include determining a temperature of a body of water by obtaining a measurement of thermal radiation from at least a portion of the body of water. The temperature of at least the portion of the body of water is then determined from the thermal radiation measurement by applying an algorithm relating the measurement to the temperature. The determined temperature can be output in various ways, including as numerical data and as graphical data, such as a temperature map of the body of water.

### CROSS-REFERENCE TO RELATED APPLICATIONS

This application claims the benefit of U.S. Provisional Application No. 61/671,410, filed on Jul. 13, 2012. The entire disclosure of the above application is incorporated herein by reference.

### FIELD

The present technology relates to measuring and monitoring water temperature.

### INTRODUCTION

This section provides background information related to the present disclosure which is not necessarily prior art.

Water temperature has long been one of the most important water quality parameters for scientists, engineers, and other professionals studying water bodies and ecosystems. Even the slightest change in water temperature can kill fish and fish eggs, and may increase algae blooms in a water body. Capturing water temperature readings can be very difficult as temperature can drastically change spatially due to many factors such as depth, sunlight exposure, distance from shoreline, and other inputs. Not understanding the full extent of the temperature changes could lead to overestimations or underestimations of the causes, and thus result in the use of improper remedies for adjusting water temperature. This is why it is important to be able to characterize a water body as a whole. Traditionally, such characterization can be limited by factors such as large surface areas, time constraints, available manpower, access to sample collection points, and project cost or budget constraints.

Temperature sampling is presently accomplished by either going out into the field with a probe and collecting samples around the body of water, or setting up monitoring stations with probes which requires people to go out to and periodically download the data collected. Both methods require people being out in the field, and most likely the entire body of water is not sampled with either method. There are also large costs associated with sending people out into the field to collect samples or data. There is the cost for getting to the site, getting a watercraft into the water, running the watercraft, and costs related to the sensors themselves.

National Oceanic and Atmospheric Administration (NOAA) National Data Buoy Center (NDBC) buoys have proven helpful in providing temperature data for a body of water, but the number and location of buoys are in constant flux. Furthermore, only select NDBC buoys have temperature sensors, and some of the buoys have sensors that measure at depths well below the surface of the body of water. Accurate temperature measurements can therefore only be obtained for small portions of a body of water that are around such buoys. This has left a need for an efficient and cost effective method for determining the temperature of an entire body of water.

The National Aeronautics and Space Administration (NASA) employs a method of determining water temperature using LANDSAT digital images of the Earth. Details of the NASA method are available online at [LANDSAThandbook.gsfc.nasa.gov/pdfs/L5_cal_document.pdf] and [LANDSAThandbook.gsfc.nasa.gov/data_prod/prog_sect11_{—}3.html], where these documents are incorporated herein by reference in their entireties. The NASA method uses a complex series of calculations based on spectral radiance determined from measurements taken from band 6 of the LANDSAT ETM+. If the measurements are taken from a high gain band of band 6, error may be introduced into the NASA method due to a more restricted dynamic range in the measurements taken. The low gain band provides an expanded dynamic range with less saturation at high Digital Number (DN) values. The expanded dynamic range provides the ability to determine temperatures across a broader range of temperatures. Conversely, the high gain band 6_{—}2 has a much more restricted dynamic range. Accordingly, error is introduced in broader temperature ranges by use of the high gain band. Spectral radiance is determined using data collected from the LANDSAT ETM+ that has been normalized. Normalization of the data may introduce error into the spectral radiance calculation due to assumptions, constants, and approximations used to normalize the data. Spectral radiance is then used to determine the effective at-satellite temperatures of the viewed area under an assumption of unity emissivity and using pre-launch calibration constants. Again, such an assumption and use of calibration constants may further introduce error into the determined temperature using the NASA method.

There is a need for a more accurate, efficient, and cost effective method for determining the temperature of an entire body of water.

### SUMMARY

The present technology includes systems, processes, articles of manufacture, and compositions that relate to monitoring water temperature.

In some embodiments, a method of determining a temperature of a body of water is provided. The method includes obtaining a measurement of thermal radiation from at least a portion of the body of water. Next, the temperature of at least the portion of the body of water is determined from the thermal radiation measurement by applying an algorithm relating the measurement to the temperature. The determined temperature is then provided as output. For example, the algorithm can be defined by X=b+m×R, where X is the determined temperature of the water, b is about −27.7, m is about 0.348, and R is the value of the thermal radiation in LANDSAT ETM+ band 6_{—}2.

In further embodiments, a method of translating a thermal image of a body of water to a temperature map is provided. The method includes processing at least a portion of the thermal image of the body of water by applying an algorithm relating the portion of the thermal image to a temperature. The temperature is then mapped in relation to the thermal image of the body of water.

In still further embodiments, a method of identifying a temperature change in a body of water is provided. A first measurement of thermal radiation is obtained from at least a portion of the body of water at a first time. A first temperature of at least the portion of the body of water is determined from the first thermal radiation measurement by applying an algorithm relating the first measurement to temperature. A second measurement of thermal radiation is obtained from at least the portion of the body of water at a second time. A second temperature of at least the portion of the body of water is determined from the second thermal radiation measurement by applying the algorithm relating the second measurement to temperature. The first temperature and the second temperature are compared to determine the temperature change, which can be provided as output.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The drawings described herein are for illustrative purposes only of selected embodiments and not all possible implementations, and are not intended to limit the scope of the present disclosure.

FIG. 1 is a drawing of a portion of North America, including the entire contiguous United States, southern Canada, and northern Mexico, showing the locations of all National Data Buoy Center (NDBC) buoys used for testing an embodiment of the present technology.

FIG. 2 is a graph of actual NDBC buoy measured water temperatures and determined water temperatures using an embodiment of the present technology.

FIG. 3 is a photograph with the temperature of the water of Lake Erie west of Lorain, Ohio in varying colors to indicate determined water temperature according to an embodiment of the present technology.

FIG. 4 is a photograph with the temperature of the water of the Atlantic Ocean near Edisto Island near South Carolina in varying colors to indicate determined water temperature according to an embodiment of the present technology.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding the methods disclosed, the order of the steps presented is exemplary in nature, and thus, the order of the steps can be different in various embodiments. Except in the examples, or where otherwise expressly indicated, all numerical quantities in this description indicating amounts of material or conditions of reaction and/or use are to be understood as modified by the word “about” in describing the broadest scope of the technology.

The present technology monitors water temperature using thermal radiation emitted from a body of water, including fresh, brackish, or salt water. A measurement of thermal radiation from the water is obtained. For example, the measurement of thermal radiation can include a thermal image captured with a thermal infrared sensor. A temperature of the water is determined from the thermal radiation measurement by applying an algorithm relating the thermal radiation measurement to the temperature. A temperature map of the body of water can be provided in this manner.

The present technology employs one or more algorithms to accurately and efficiently determine the temperature of a body of water. The algorithms were developed and validated using National Data Buoy Center (NDBC) buoy water temperature measurement data in order to correlate LANDSAT digital images and thermal radiation measurements to surface water temperature. Such images and thermal radiation measurements include those obtained using the LANDSAT 7 Enhanced Thematic Mapper Plus (ETM+) and/or using the LANDSAT 8 Thermal InfraRed Sensor (TIRS). LANDSAT satellites continuously acquire space-based images of the Earth's land surface, coastal shallows, and coral reefs. The LANDSAT Program, a joint effort of the U.S. Geological Survey (USGS) and the National Aeronautics and Space Administration (NASA), was established to routinely gather imagery from space. NASA develops the remote-sensing instruments and spacecraft, then launches and validates the performance of the instruments and satellites. The USGS then assumes ownership and operation of the satellites, in addition to managing all ground reception, data archiving, product generation, and distribution. The result of this program is a long-term record of natural and human-induced changes on the global landscape.

LANDSAT satellites image the Earth's surface along the satellite's ground track in a 185-kilometer-wide (115-mile-wide) swath as the satellite moves in a descending orbit (moving from north to south) over the sunlit side of the Earth. LANDSAT 7 and LANDSAT 8 orbit the Earth at 705 kilometers (438 miles) altitude. They each make a complete orbit every 99 minutes, complete about 14 full orbits each day, and cross every point on Earth once every 16 days. Although each satellite has a 16-day full-Earth-coverage cycle, their orbits are offset to allow 8-day repeat coverage of any LANDSAT scene area on the globe.

The primary sensor onboard LAND SATS 1, 2, and 3 was the Multispectral Scanner (MSS), with an image resolution of approximately 80 meters in four spectral bands ranging from the visible green to the near-infrared (IR) wavelengths. The improved Thematic Mapper (TM) sensors onboard LANDSATS 4 and 5 were designed with several additional bands in the shortwave infrared (SWIR) part of the spectrum; improved spatial resolution of 30 meters for the visible, near-IR, and SWIR bands; and the addition of a 120-meter thermal-IR band. LANDSAT 7 carries the Enhanced Thematic Mapper Plus (ETM+), with 30-meter visible, near-IR, and SWIR bands, a 60-meter thermal band, and a 15-meter panchromatic band. LANDSAT 8, launched on Feb. 11, 2013, ensures the continued acquisition and availability of LANDSAT data, which will be consistent with current standard LANDSAT data products. About 400 scenes are acquired each day. All scenes are processed to data products and are available for download within 24 hours of reception and archiving.

LANDSAT 8 carries two push-broom sensors, the Operational Land Imager (OLI) and Thermal Infrared Sensor (TIRS), both of which provide improved signal to noise ratio and 12-bit radiometric quantization of the data. The OLI collects data in nine shortwave bands—eight spectral bands at 30-meter resolution and one panchromatic band at 15 meters. Refined heritage bands and the addition of a new coastal/aerosol band, as well as a new cirrus band, creates data products with improved radiometric performance. OLI data products have a 16-bit range. A new quality assurance band provides information on the presence of features such as clouds and terrain occlusion. The TIRS captures data in two long wave thermal bands with 100-meter resolution, and is registered to and delivered with the OLI data as a single product. TIRS data products have a 30-meter resolution and a 16-bit range.

With respect to processing thermal radiation measurements from LANDSAT images, LANDSAT 7 data is in an 8 bit format while LANDSAT 8 data is in a 16 bit format. In order to use LANDSAT 8 data with a LANDSAT 7 algorithm, for example, the LANDSAT 7 algorithm can simply be resealed by a ratio of 256/65,536. In this manner, the present technology can employ thermal radiation measurements obtained from LANDSAT 7, LANDSAT 8, or both LANDSAT 7 and LANDSAT 8.

It should be further noted, however, that the present technology may be carried out using a measurement of thermal radiation from a body of water of interest regardless of how the measurement of thermal radiation was obtained. In particular, the measurement of thermal radiation can include a thermal image captured with a thermal infrared sensor from various sources. As already described, LANDSAT 7 and LANDSAT 8 are examples of two such sources of thermal radiation measurements. Other sources include those acquired from other remote sensing platforms in space, various atmospheric aerial sources such as various manned and unmanned aircraft, including airplanes, helicopters, balloons, etc., as well as elevated terrestrial-based sources, such as towers, buildings, or other various artificial or natural geographically elevated vantage points with respect to the body of water of interest.

The present technology was developed by obtaining measurements of thermal radiation, relating the thermal radiation measurements to actual temperature measurements, and producing an algorithm that translates a thermal radiation measurement to a temperature measurement. To develop the algorithm, actual temperature measurements were obtained using NDBC buoys. Only NDBC buoys containing water temperature sensors were selected, as not all NDBC buoys contain such sensors. The NDBC buoys were also selected to be at least sixty (60) meters from the shoreline to ensure that the entire buoy temperature measurement is not affected by shoreline temperature effects. NDBC buoys were selected in warm water climates and cold water climates to ensure that the developed algorithm was accurate across various temperature ranges. Further, the selected buoys included sensors that measure water temperatures at a depth no deeper than one (1) meter. Buoys were also selected in fresh water and salt water to eliminate salinity as a factor in the temperature determined by the algorithm.

Once the NDBC buoys were selected in accordance with the criteria described hereinabove, LANDSAT 7 ETM+ data was accumulated from high gain band 6_{—}2 (the 2^{nd} band of high gain band 6) measurements of thermal radiation during satellite overpass(es) of the body of water at each buoy location. The data from the overpass(es) was culled to leave only the data taken within one hour of a temperature reading by each NDBC buoy. This is important as water moves over time and the temperature may change. The data collected from the LANDSAT 7 ETM+ satellite was then compared to the NDBC buoy data to develop an algorithm whereby the thermal radiation readings measured by the LANDSAT 7 ETM+ satellite can be simply and efficiently converted to determine a temperature measurement of a body of water over which the satellite passes.

In particular, various measurements of thermal radiation from LANDSAT data at a various buoy locations were matched with actual temperature values from the buoys. These matched values were then subjected to first order linear regression to provide a best fit of the various matched values. An equation was derived based on the fit, the equation being of the form X=b+m×R, where X is the temperature of the water, b is a constant, m is the slope, and R is the measurement of thermal radiation in LANDSAT ETM+ band 6_{—}2. The developed algorithm therefore allows the temperature of a body of water to be determined without having to collect data from individual buoys that may not read the entire body of water. In this way, a temperature or a temperature map can be determined for a portion of a body of water or a whole body of water based solely on thermal radiation measurements. Using the equation, buoy temperatures or actual temperature measurements are no longer necessary to accurately determine water temperature.

An algorithm according to an embodiment of the present technology is as follows: X=b+m×R; wherein X is the determined temperature of the water; b is about −27.7; m is about 0.348; and R is the measurement of thermal radiation in LANDSAT 7 ETM+ band 6_{—}2. The algorithm can also presented as X=−27.7+0.348×R, where X is the determined temperature of the water and R is the measurement of thermal radiation in LANDSAT ETM+ band 6_{—}2.

As previously described, LANDSAT 7 data is in an 8 bit format while LANDSAT 8 data is in a 16 bit format. The coefficients presented in the above algorithm are for 8 bit data from LANDSAT 7. In order to use LANDSAT 8 with the above algorithm, the coefficients can be resealed by the ratio 256/65,536 times the coefficient. In this manner, the current algorithm can be used with both LANDSAT 7 and LANDSAT 8. The present technology can also be applied using only LANDSAT 8 data to develop an algorithm with coefficients tailored specifically to LANDSAT 8. The coefficients of such a LANDSAT 8 algorithm can be back-converted from 16 bit format to 8 bit format in a similar fashion for use with LANDSAT 7.

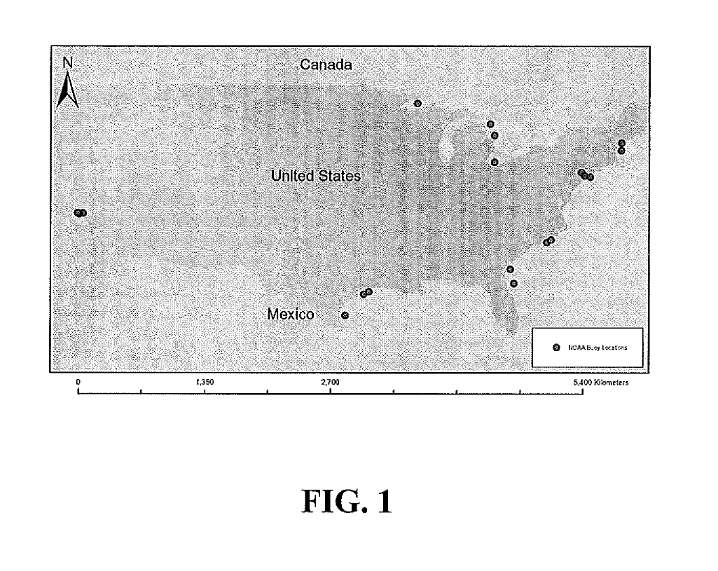

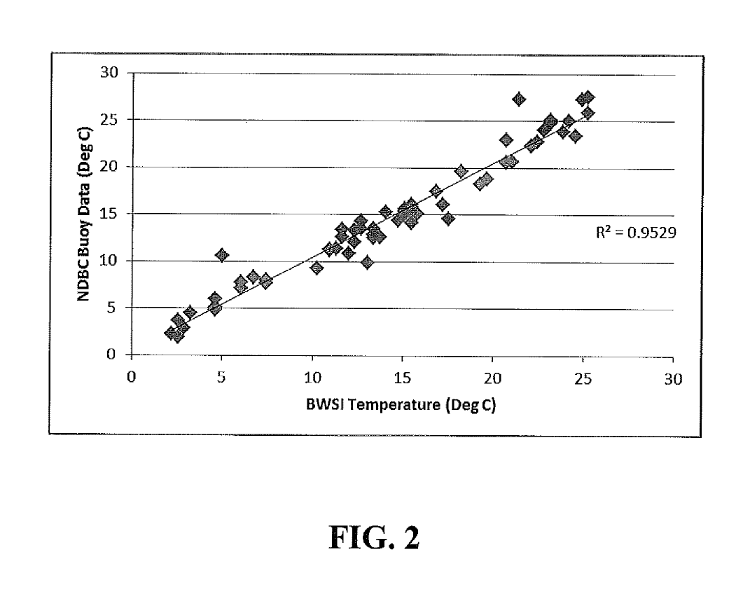

Using the steps described above for developing the algorithm, water temperature data from the selected NDBC buoys (see FIG. 1) was compared to water temperatures determined using the algorithm. As shown in FIG. 2, the results showed a very high correlation between the determined temperatures and the actual temperatures. The correlation is quite high (coefficient of determination, r²=0.953) showing that the algorithm correctly determines temperatures across a wide range of temperatures and conditions.

The algorithm according to the present technology was also compared to the methods developed by NASA to determine water temperature using satellite data. In determining water temperature, satellite rendering image pixels are converted to units of absolute radiance using 32 bit floating point calculations. Pixel values are then scaled to byte values prior to media output. The following equation is used to convert to radiance units:

L_{λ}=Grescale*QCAL+Brescale

which is also expressed as:

L_{λ}=((LMAX_{λ}−LMIN_{λ})/(QCALMAX−QCALMIN))*(QCAL−CALMIN)+LMIN_{λ}

where:
L_{λ}=Spectral Radiance at the sensor's aperture in watts/(meter squared*ster*μm)
Grescale=Resealed gain (the data product “gain” contained in the Level 1 product header or ancillary data record) in watts/(meter squared*ster*μm)/DN
Brescale=Resealed bias (the data product “offset” contained in the Level 1 product header or ancillary data record) in watts/(meter squared*ster*μm)
QCAL=the quantized calibrated pixel value in DN
LMIN_{λ}=the spectral radiance that is scaled to QCALMIN in watts/(meter squared*ster*μm)
LMAX_{λ}=the spectral radiance that is scaled to QCALMAX in watts/(meter squared*ster*μm)
QCALMIN=the minimum quantized calibrated pixel value (corresponding to LMIN_{λ}) in DN
   =1 for LPGS products
   =1 for NLAPS products processed after Apr. 4, 2004
   =0 for NLAPS products processed before Apr. 5, 2004
QCALMAX=the maximum quantized calibrated pixel value (corresponding to LMAX_{λ}) in DN
   =255

Spectral radiance L_{λ} is then converted to temperature using the following equation:

$$T = \frac{K 2}{ln ( \frac{K 1}{{L}_{λ}} + 1 )}$$

where:
   T=Effective at-satellite temperature in Kelvin
   K2=Calibration constant 2 from Table 11.5
   K1=Calibration constant 1 from Table 11.5
   L=Spectral radiance in watts/(meter squared*ster*μm)

Next, water temperature measurements determined using the algorithm according to the present technology at select buoy locations were compared to water temperature measurements determined using the NASA method at the same buoy locations. Table 2 shows data from the algorithm developed in accordance with the present technology as compared to data using the NASA method, both of which are compared against actual NDBC buoy measurements.

TABLE 2

| Buoy | | Buoy Temp | Inventive Temp Alg | NASA Alg |
|---|---|---|---|---|
| Station | Date | (deg C.) | (deg C.) | (deg C.) |
| 45006 | May 26, 2003 | 1.9 | 2.576 | 5.690 |
| 45003 | May 18, 2007 | 2.3 | 2.228 | 5.343 |
| 45008 | May 18, 2007 | 2.9 | 2.924 | 6.035 |
| 44007 | Apr. 3, 2011 | 3.7 | 2.576 | 5.690 |
| 44095 | Apr. 3, 2011 | 4.5 | 3.272 | 6.380 |
| 44025 | Apr. 9, 2005 | 4.8 | 4.664 | 7.746 |
| 44025 | Mar. 11, 2006 | 5.1 | 4.664 | 7.746 |
| 44025 | Feb. 7, 2006 | 6 | 4.664 | 8.085 |
| 44025 | Jan. 1, 2004 | 7.1 | 6.056 | 9.095 |
| 44025 | Apr. 17, 2008 | 7.7 | 7.448 | 10.427 |
| 44065 | Dec. 29, 2011 | 7.8 | 6.056 | 9.052 |
| 45006 | Oct. 30, 2008 | 8 | 7.448 | 10.758 |
| 44025 | Dec. 29, 2011 | 8.3 | 6.752 | 9.684 |
| 45006 | Jul. 26, 2008 | 9.3 | 10.232 | 13.044 |
| 45003 | Jun. 24, 2009 | 9.9 | 13.016 | 15.599 |
| 45005 | Apr. 1, 2010 | 10.6 | 5.012 | 8.085 |
| 46092 | May 5, 2011 | 10.9 | 11.972 | 14.648 |
| 46042 | May 21, 2011 | 11.2 | 10.928 | 13.688 |
| 46092 | May 21, 2011 | 11.3 | 10.928 | 14.009 |
| 46236 | May 21, 2011 | 11.4 | 11.276 | 14.009 |
| 46236 | May 5, 2011 | 12.1 | 12.32 | 14.966 |
| 46042 | May 5, 2011 | 12.5 | 13.364 | 16.229 |
| 44025 | Nov. 19, 2011 | 12.6 | 11.624 | 14.323 |
| 45006 | Sep. 12, 2008 | 12.6 | 13.712 | 16.229 |
| 44025 | Nov. 3, 2011 | 12.9 | 13.364 | 15.907 |
| 46092 | Oct. 28, 2011 | 13.3 | 12.32 | 15.283 |
| 44065 | Nov. 11, 2008 | 13.4 | 11.624 | 14.263 |
| 45003 | Jul. 21, 2007 | 13.5 | 13.364 | 15.915 |
| 46236 | Oct. 28, 2011 | 13.5 | 12.668 | 15.283 |
| 44025 | May 30, 2006 | 14.1 | 15.452 | 17.789 |
| 44025 | Nov. 11, 2008 | 14.3 | 12.668 | 15.293 |
| 46114 | Oct. 12, 2011 | 14.4 | 14.756 | 17.168 |
| 45008 | Jun. 24, 2009 | 14.6 | 17.54 | 19.634 |
| 46042 | Oct. 12, 2011 | 15 | 15.104 | 17.479 |
| 44007 | Jun. 19, 2010 | 15.1 | 15.8 | 18.099 |
| 46042 | Sep. 26, 2011 | 15.3 | 14.06 | 16.543 |
| 46114 | Oct. 28, 2011 | 15.4 | 15.104 | 17.479 |
| 46042 | Oct. 28, 2011 | 15.6 | 15.104 | 17.479 |
| 45006 | Aug. 11, 2008 | 15.8 | 15.452 | 17.789 |
| 44095 | Jun. 19, 2010 | 16.1 | 17.192 | 19.329 |
| 46236 | Oct. 12, 2011 | 16.1 | 15.452 | 17.789 |
| 44025 | Jun. 18, 2007 | 17.5 | 16.844 | 19.023 |
| 45005 | Jun. 6, 2005 | 18.3 | 19.28 | 21.150 |
| 44025 | Jul. 1, 2006 | 18.8 | 19.628 | 21.450 |
| 44025 | Sep. 8, 2008 | 19.6 | 18.236 | 20.243 |
| 45005 | Sep. 5, 2009 | 20.6 | 20.672 | 22.348 |
| 45005 | Jun. 20, 2010 | 20.7 | 21.02 | 22.348 |
| 41036 | Oct. 26, 2011 | 22.4 | 22.064 | 23.535 |
| 41008 | Oct. 24, 2011 | 22.8 | 22.412 | 23.830 |
| 45005 | Jul. 8, 2005 | 23 | 20.672 | 22.348 |
| 44025 | Jul. 17, 2006 | 23.4 | 24.5 | 25.584 |
| 45005 | Jul. 9, 2011 | 23.9 | 23.804 | 24.710 |
| 41037 | Oct. 26, 2011 | 24 | 22.76 | 24.124 |
| 41036 | Jun. 4, 2011 | 24.8 | 23.108 | 24.417 |
| 44065 | Jul. 31, 2011 | 25 | 24.152 | 25.294 |
| 45005 | Aug. 7, 2010 | 25.1 | 23.108 | 24.417 |
| 41036 | Jun. 20, 2011 | 25.9 | 25.196 | 26.164 |
| 41012 | Jun. 15, 2010 | 27.3 | 24.848 | 25.580 |
| 41036 | Jul. 22, 2011 | 27.3 | 21.368 | 22.943 |
| 41008 | Jul. 20, 2011 | 27.6 | 25.196 | 25.872 |
| | | Standard | | |
| | | Error | 1.5 | 1.6 |

The standard error for the algorithm of the present technology was about 1.5, while the standard error for the NASA method was 1.6. That is, the instant algorithm is over 6% more accurate than the NASA method. Accordingly, the algorithm according to the present technology provides more accurate and meaningful temperature data over an entire body of water more efficiently, quickly, and easily than any methods known in the art.

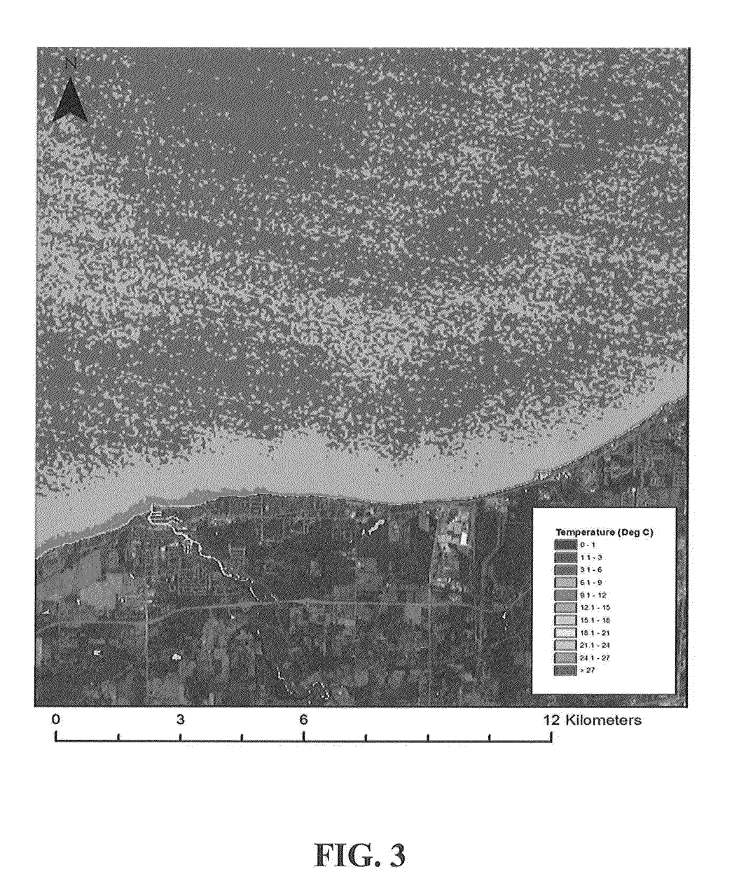

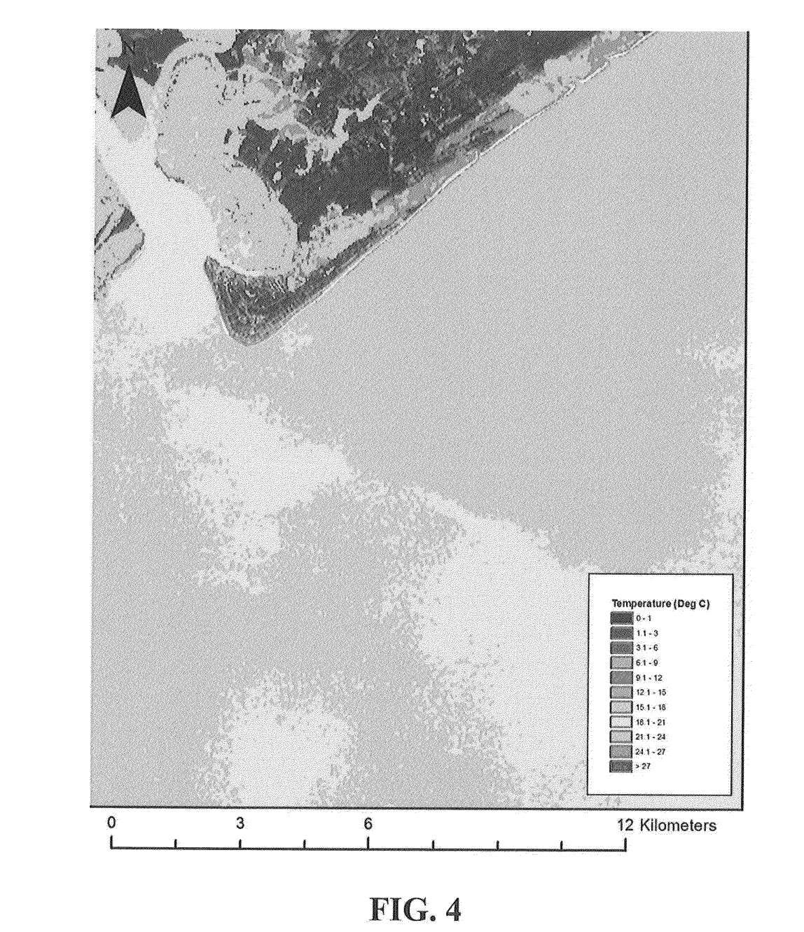

The present technology also includes a system using an algorithm for converting LANDSAT ETM+ measurements into reports and/or images showing water temperature over an entire body of water. The images may be any digital image, a digital image with color coding, such as those found in FIGS. 3 and 4, and/or a GeoTIFF including the determined temperature of the water and the coordinates of the determined temperature location.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. A method of determining a temperature of a body of water comprising:
obtaining a thermal radiation measurement from at least a portion of the body of water;
determining a temperature of at least the portion of the body of water from the thermal radiation measurement by applying an algorithm relating the thermal radiation measurement to the temperature of at least the portion of the body of water, wherein the algorithm is the result of fitting one of a line and a curve to actual water temperature data relative to measurements of thermal radiation; and
outputting the determined temperature.

2. The method of claim 1, wherein the thermal radiation measurement includes a thermal image captured with a thermal infrared sensor.

3. The method of claim 1, wherein the thermal radiation measurement includes a measurement obtained from the LANDSAT 7 Enhanced Thematic Mapper Plus (ETM+).

4. The method according to claim 3, wherein the thermal radiation measurement includes a measurement taken from high gain band 6_{—}2 of the LANDSAT ETM+.

5. The method of claim 1, wherein the thermal radiation measurement includes a measurement obtained from the LANDSAT 8 Thermal InfraRed Sensor (TIRS).

6. The method of claim 1, wherein the thermal radiation measurement includes a measurement obtained from the LANDSAT 7 ETM+ and a measurement obtained from the LANDSAT 8 TIRS.

7. The method of claim 1, wherein the body of water is one of fresh water, brackish water, and salt water.

8. The method of claim 1, wherein the algorithm is a linear equation.

9. The method of claim 1, wherein the algorithm is X=b+m×R;
X is the determined temperature of the water;
b is about −27.7;
m is about 0.348; and
R is the measurement of thermal radiation in LANDSAT ETM+ band 6_{—}2.

10. The method of claim 9, wherein the thermal radiation measurement includes a measurement in a format other than 8 bit format and the algorithm is resealed to 8 bit format to determine the temperature of the thermal radiation measurement in a format other than 8 bit format.

11. The method of claim 9, wherein the thermal radiation measurement includes a measurement obtained from the LANDSAT 8 TIRS in a 16 bit format and the algorithm is resealed by a ratio of 256/65,536.

12. The method according to claim 1, wherein outputting the determined temperature includes transmitting the determined temperature to a remote location.

13. The method according to claim 1, wherein outputting the determined temperature includes generating a report of the determined temperature of at least the portion of the body of water.

14. The method according to claim 13, wherein the report is one of an image file and a GeoTIFF including the determined temperature of the water and the coordinates of the determined temperature location.

15. A method of monitoring a temperature of a body of water comprising:
receiving a temperature of the body of water, wherein the temperature was determined by a method comprising:
obtaining a thermal radiation measurement from at least a portion of the body of water;
determining a temperature of at least the portion of the body of water from the thermal radiation measurement by applying an algorithm relating the thermal radiation measurement to the temperature of at least the portion of the body of water, wherein the algorithm is the result of fitting one of a line and a curve to actual water temperature data relative to measurements of thermal radiation; and
outputting the determined temperature.

16. The method of claim 15, wherein the algorithm is X=b+m×R;
X is the temperature;
b is about −27.7;
m is about 0.348; and
R is the measurement of thermal radiation in LANDSAT ETM+ band 6_{—}2.

17. The method of claim 16, wherein the thermal radiation measurement includes a measurement in a format other than 8 bit format and the algorithm is resealed to 8 bit format to determine the temperature of the thermal radiation measurement in a format other than 8 bit format.

18. The method of claim 16, wherein the thermal radiation measurement includes a measurement obtained from the LANDSAT 8 TIRS in a 16 bit format and the algorithm is resealed by a ratio of 256/65,536.

19. A method of translating a thermal image of a body of water to a temperature map comprising:
processing at least a portion of the thermal image of the body of water by applying an algorithm relating the portion of the thermal image to a temperature, wherein the algorithm is the result of fitting one of a line and a curve to actual water temperature data relative to measurements of thermal radiation; and
mapping the temperature in relation to the thermal image of the body of water.

20. A method of identifying a temperature change in a body of water comprising:
obtaining a first thermal radiation measurement from at least a portion of the body of water at a first time;
determining a first temperature of at least the portion of the body of water from the first thermal radiation measurement by applying an algorithm relating the first thermal radiation measurement to temperature, wherein the algorithm is the result of fitting one of a line and a curve to actual water temperature data relative to measurements of thermal radiation;
obtaining a second thermal radiation measurement from at least the portion of the body of water at a second time;
determining a second temperature of at least the portion of the body of water from the second thermal radiation measurement by applying the algorithm relating the second thermal radiation measurement to temperature;
comparing the first temperature and the second temperature to determine the temperature change; and
outputting the temperature change.
